(19) United States
(12) Patent Application Publication
(10) Pub. No.: US 2006/0205994 A1
(43) Pub. Date: Sep. 14, 2006

(54) Vibrational delta and theta brain wave induction apparatus and method for the stimulation of sleep

(76) Inventor: Gerard V. Sunnen, New York, NY (US)

Correspondence Address: OSTROLENK FABER GERB & SOFFEN, 1180 AVENUE OF THE AMERICAS, NEW YORK, NY, 100368403, US

(21) Appl. No.: 11/075,075

(22) Filed: Mar. 8, 2005

(51) Int. Cl. A61M 21/00

(52) U.S. Cl. 600/27

(57) ABSTRACT

An apparatus and method for generating sleep-inducing stimuli, including a programmable controller operable for generating a sleep-inducing rhythm; and a transducer unit containing at least one transducer connected to the controller for receiving the sleep-inducing rhythm and generating and applying the sleep-inducing stimuli to a user in accordance with the rhythm. The stimuli may be in the form of vibration, light, sound, and/or electrical current. The stimuli are adapted to induce alpha, theta, and/or delta brain waves.

### BACKGROUND OF THE INVENTION

Sleep disorders are increasing in their incidence and prevalence in the general population and pose enormous public health issues. An effective therapy for the most common sleep disorders could bring massive public health benefits in terms of physical health, psychological well-being, and productivity. Indeed, sleep disorders have been strongly associated with depression and anxiety.

The normal sleep cycle is divided into two main phases each embodying characteristic physiological constellations.

Sleep centers in the nervous system regulate the rhythm of circadian sleep/wake cycles. The suprachiasmatic (SNC) nucleus of the anterior hypothalamus receives impulses from retinal nerves and other special senses. The SNC projects nerve fibers into the hypothalamus which, in turn, has influence over locomotor activity, food and water intake, body temperature, and hormone levels. It is apparent, therefore, that sleep functions actively weave into the spectrum of basic bodily functions.

In one of these phases, a salient feature is the emergence of rapid eye movement (REM). This phase is called REM sleep. In the other phase, eye movements are relatively absent. This phase is called non-REM, or NREM sleep. During the course of the night, there are several periods of alternating REM and NREM sleep phases.

REM periods are associated with, in addition to eye motions, EEG rhythms found in the waking state such as alpha waves (8 to 12 cycles per second), the inhibition of muscle activity, the engagement of the autonomic nervous system as expressed in blood pressure and heart rate fluctuations, and with dreaming. 20% to 25% of sleep time is devoted to REM sleep. In normal sleep, an initial NREM phase of approximately 70 to 100 minutes duration is followed by the first REM period. Depending upon total sleep time, this cycle is repeated 4 to 6 times during the night.

NREM cycles do not show the characteristic horizontal and vertical eye motions found in REM sleep. In the beginning of the nightly sleep cycle, alpha waves begin to give way to low-voltage, theta, 4 to 7 cycles per second brain waves. This is identified as stage 1 sleep.

Stage 2 sleep usually occurs less than a minute later, but may be delayed for several minutes. 12 to 14 cycle spindle tracings appear with occasional slow triphasic waves, known as K complexes.

Soon thereafter, cycles ranging from 4 to 0.5 cycles per second appear. These are known as delta waves. When occupying less than 50% of the tracing, this is designated as stage 3 sleep. When delta waves account for more than 50% of the EEG tracing, stage 4 is achieved. Taken together, stages 3 and 4 are known as delta sleep or slow wave sleep (SWS).

In the transition from full wakefulness to somnolence, drowsiness, slumber, and finally sleep, waking EEG patterns give way to stages 1 through 4 NREM sleep. Delta sleep provides the most recuperative, highly quality sleep.

Individuals afflicted with initial insomnia have difficulties transitioning from the waking state to stages 1 and 2, and on to stages 3 and 4.

### SUMMARY OF THE INVENTION

This invention aims to assist in the therapy of the most common manifestation of sleep dysfunction, namely the difficulty in falling asleep, so-called initial insomnia.

This invention proposes to prompt sleep-generating brain waves in brain sleep centers by stimulating sensory organs such as the skin, the visual senses, and the auditory senses with delta rhythms.

The delta wave prompting, importantly, is preferably individualized. Indeed, individuals show important variations in their preferences for the frequencies and the properties of the vibrations, and their translation into sound and color.

The invention's rationale is based upon electroencephalographic (EEG) studies that have delineated, with ever-increasing precision, the architecture of normal and abnormal sleep patterns. It is also inspired by a unifying principle applicable to the nervous system: Every neuron in the nervous system finds connections to every other neuron.

This principle forms the foundation of this invention. Specifically, a stimulus applied to the skin, such as a vibration, will travel through nervous system networks, eventually impacting upon and resonating into all cortical and subcortical structures, including the sleep centers.

The initial stages of sleep show characteristic brain wave configurations, namely slow waves and delta waves. Encephalographically-speaking, individuals afflicted with initial insomnia have difficulties in making the transition from waking brain wave patterns to patterns associated with the onset of normal sleep.

The present invention seeks to encourage this transition using vibrational prompting. In addition, the apparatus and method also comprise optional auditory, visual, and electrophysiological stimulation to impel the waking brain into adopting theta, and eventually, delta brain wave configurations.

Neurophysiological prompting is a process by which an external stimulus acts as an inducer for a desired physiological response. In this case, the desired response is the generation and the maintenance of delta sleep waves.

In order to facilitate the transition from the waking state to sleep, this invention uses vibrational prompting synchronized to theta and delta wave frequencies. This vibrational prompting may be supplemented or supplanted by auditory, visual, and subliminal or para-subliminal electro-physiological stimulation.

The ability to perceive vibrational stimulation is called pallesthesia. Receptors in the skin and deeper tissues, including Pacinian corpuscules, relay their messages to the dorsal columns of the spinal cord, making their way to the thalamus and from there to somesthetic cortical areas for detailed recognition.

Along this trajectory, communications are made with multiple areas of the brain, including deeper structures. These include the hypothalamic sleep centers. In addition, once having reached the somesthetic cortex, vibrational impulses freely extend their reach into other cortical areas including the frontal, temporal, and occipital lobes. For example, it is appreciated that vibrations applied to the skin may be perceived visually.

Synesthesia is the phenomenon which describes such cross-sensory perception. The significance of this cross-sensory phenomenon is that, for example, a vibrational stimulus applied anywhere on the body will, given adequate time and repeated applications, create neural reverberations into many areas of the nervous system. If this vibrational stimulus is given a delta frequency, the effect will eventually make its way into brain sleep centers, which will be prompted to mimic this sleep-inducing rhythm.

The apparatus generates a desired brain wave frequency through a microprocessor unit. The frequency may be selected by the user, or may be predetermined. Thus, the unit frequency setting may be set anywhere from 8 to ½ cycles per second. Some individuals find that inducing theta waves (8 to 4 cycles per second) automatically paves the way for delta wave production. Others will prompt delta waves from the start.

Other options are possible. The unit, for example, may emit a sequential progression of frequencies which mimic the transition from the waking state (descending from 12 to 8 cycles per second), to stage 2 sleep (descending from 8 to 4 cycles per second), then on to delta sleep, from 4 to ½ cycles per second. Each of these stages may be programmed as to their respective durations.

The frequencies generated by the microprocessor are capable of driving different modalities of stimuli, either individually, or in combination. Among them:
1. Vibration. The device is capable of driving vibrational rhythms ranging from 8 to 4 cycles per second, thus mimicking Stage 1 sleep, and from 4 to ½ cycles per second, mimicking delta sleep. Vibrations generated by the vibrator unit and imparted to the sleep pad are directly transmitted to the individual via body contact.
2. Sound. The rhythm frequency may, in addition to vibrations, drive any one of a number of sounds, or tones. A menu of pleasing sounds may be chosen such as waterfalls, waves, musical instruments, or electronically generated sounds.
3. Colored lights. The rhythm frequency may drive colored lights as well. An LED (light emitting diode) or other light source, capable of being perceived by the sleeper through closed eyelids is incorporated in the device. Color preferences may be selected. A random presentation of colors may also be selected.
4. Subliminal and para-subliminal electrophysiological stimulation. The rhythm may also drive an electrophysiological stimulation unit (ESU). This ESU may emit microcurrents below the threshold of perception, or ones barely perceptible so as not to be distracting. These currents find their way into the autonomic nervous system, ultimately influencing the brain's sleep centers.

Thus, the invention provides an apparatus and method designed to assist in the transition from wakefulness to sleep by means of theta and/or delta brain wave rhythmic prompting via vibrational stimulation applied to the skin. The apparatus is capable of supplementing or supplanting the vibrational stimulation with visual, auditory, and electrophysiological stimulation.

According to a preferred embodiment of the invention, the apparatus may comprise:
A. A flat pad of various dimensions or shapes. The illustration proposes a round pad with a diameter of 8 inches, which may or may not be made of electroconductive material, and which may show a picture suggestive of sleep. Many sizes, shapes, and designs are possible. The thickness of the pad is such that it will be comfortable to appose one's head to it. However, the user may also elect to appose the pad to other parts of the body.
B. A battery or other power supply which powers the various functions of the pad.
C. The pad incorporates a microprocessor which regulates several functions. The most fundamental function is the rhythm function which ranges from 12 to ½ cycles per second. However, the target rhythms range anywhere from 8 to ½ cycles per second, corresponding to theta (8 to 4 cps) and to delta (4 to ½ cps) brain wave rhythms. A rhythm may be pre-set, or may be assigned a sequential progression from waking brain wave rhythms (alpha, 12 to 8 cycles per second), to theta (8 to 4 cycles per second), and on to delta (4 to ½ cycles per second).
D. A digital readout and a rhythm light display showing the rhythm selected.
E. A vibrational unit capable of transmitting the selected rhythms to the pad so that the entire pad vibrates. The intensity of the vibrations may be selected by the user.
F. A mini-speaker capable of translating the rhythm into sound. Modalities of sound may be selected to manifest tones, pleasing sounds of nature such as water flowing or waves, music, or electronically generated sounds. A volume control is included.
G. A light source capable of synchronous pulsations with the rhythm. The illustration shows a circular LED light source surrounding the pad. It is strong enough so that the user will perceive it with the eyes closed. Different colors may be selected.
H. An electrophysiological unit capable of imparting subliminal and para-subliminal electrical impulses to the pad. The unit emits microcurrent impulses to the pad in synchrony with the chosen rhythm, and determines its output power. Current, measured in milliamperes (mA), may range from 1 to 100 mA. Electrical pulse width may range from 5 to 500 microseconds (μs). The pulse rate may range from 1 to 2 pulses per second (pps) to 250 pps. An electroconductive solution may be applied to the pad so that electrical impulses may be more reliably imparted to the user.
I. A timer function capable of shutting off the unit after a selected time span; or capable of re-starting the unit at selected times during the night.

A preferred embodiment of a method may provide:
A. A process by which sleep is induced via the neurophysiological prompting of theta and, more importantly, delta brain waves. The latter brain waves are associated with Stages 3 and 4 NREM deep sleep.
B. The prompting is initiated by the presentation of one or more rhythmic stimuli which, singly or in unison, are capable of initiating nervous system resonance. Stimuli include vibration, light and color, sound, and electrophysiological stimulation.
C. An option to present the individual with a constant pre-selected rhythm within the span of the theta/delta range, namely 8 to ½ cycles per second.
D. An option to present the individual with a progression of rhythms designed to mimic the normal sleep pattern, namely a progression from alpha, to theta, and finally to delta rhythms.
E. An option to select the vibrational modality only, the light and color modality only, the sound modality only, the electrophysiological modality only, and any combination thereof.
F. An option to activate the pad at different times during the night.
G. An option to have an automatic shut-off.

Other features and advantages of the present invention will become apparent from the following description of embodiments of the invention which refers to the accompanying drawings.

### BRIEF DESCRIPTION OF THE DRAWINGS

FIG. 1 is a top view of a sleep pad according to an embodiment of the invention.

FIG. 2 is a side view of the sleep pad.

FIG. 3 is a top view of the sleep pad with its top surface removed.

### DETAILED DESCRIPTION OF AN EMBODIMENT OF THE INVENTION

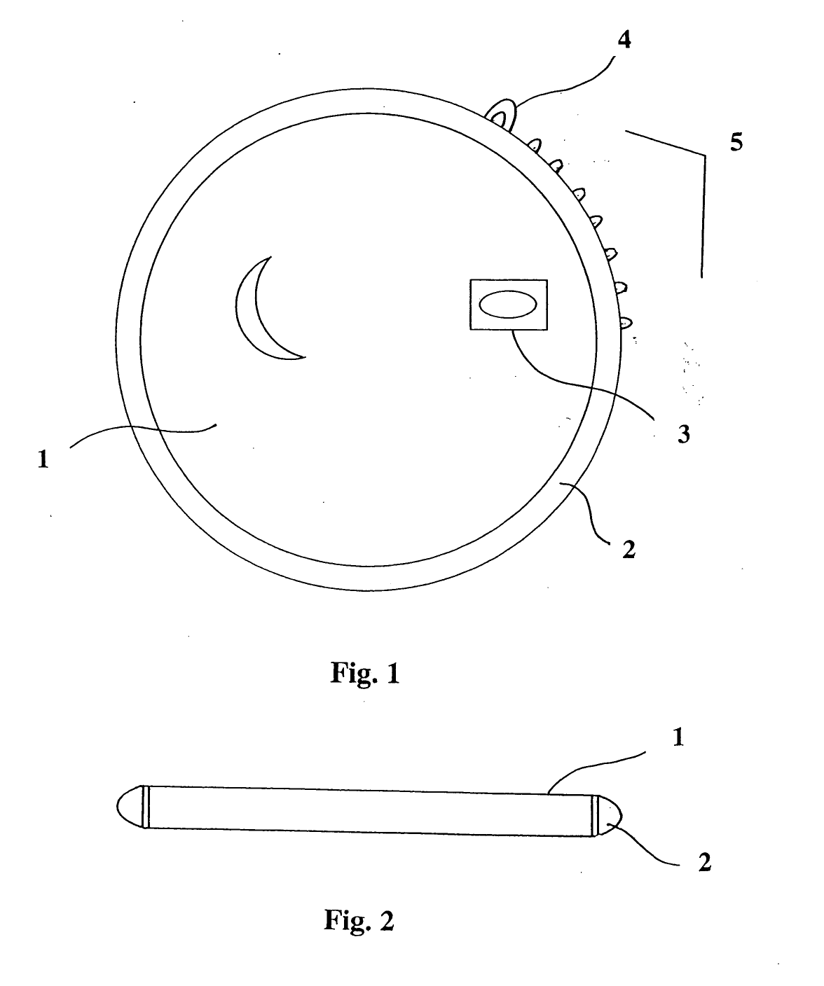

FIG. 1 shows the sleep pad in its frontal view. It shows a circular device perhaps 8 inches in diameter, optionally adorned with a design suggestive of sleep on its surface (1). The other visible component is an LED array (2) around the rim, an LCD (liquid crystal display) (3), a rhythm light display (4), and programming buttons (5).

FIG. 2 shows a lateral view of the sleep device with the surface element (1) and the LED (light emitting diode) array (2) on the outer edge.

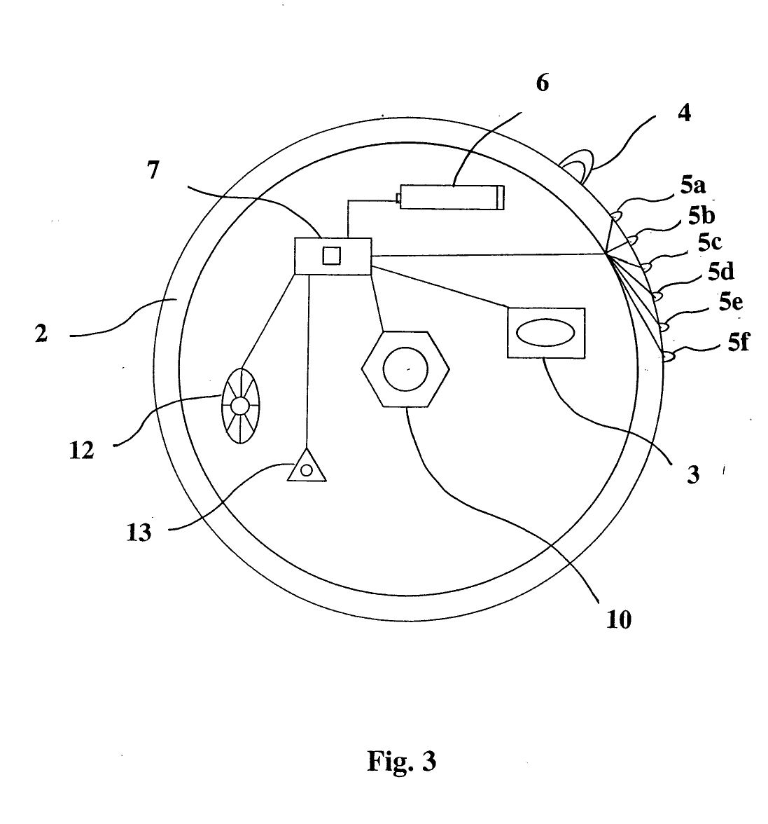

FIG. 3 shows the sleep pad without the surface element. Uncovered are the working elements of the sleep device including the battery (6) which energizes the microprocessor (7). The microprocessor is programmable via the control buttons (5a), (5b), (5c), (5d), (5e), and (5f). Button (5a) programs the rhythm function which is both displayed on the LCD (liquid crystal display) (3), and the rhythm light display (4). Button (5b) programs the vibrational unit (10). Button (5c) programs the speaker (12). Button (5d) programs the LED (light emitting diode) array (2). Button (5e) programs the electrophysiological unit (13). Button (5f) programs the timing function of the microprocessor (7).

The programmable microprocessor has a variety of functions. Foremost is the rhythm function programmed through control (5a). The rhythm selection is shown on the LCD display (3). The rhythm control may select a fixed rhythm, or may select a sequence of rhythms, such as a rhythm progression from alpha (12 to 8 cycles per second), to theta (8 to 4 cycles per second), on to delta (4 to ½ cycles per second), for example.

The respective rhythms may be generated for variable corresponding lengths of time. The timer control (5f) programs the desired time parameters of the unit including automatic shut off and re-start.

The apparatus presents as a flat pad made of pliable and electroconductive material, such as, for example, carbon silicone. It is thin and comfortable enough to rest one's head upon it. Yet, it may be apposed to any part of the body. In the illustration in FIG. 1, this pad is 8 inches in diameter and is adorned with a design suggestive of sleep. The pad, however, may adopt any one of a number of different sizes, configurations, and designs.

The function of the sleep pad of greatest therapeutic value is believed to be its vibrational capacity. However, in addition, it has the capacity, predicated upon individual choice or therapeutic preference, to express rhythmic light, rhythmic sound, and rhythmic electrophysiological stimuli.

The sleep pad is provided with an energy source, a battery, FIG. 3 (6) This battery powers a microprocessor, FIG. 3 (7), which regulates all the functions of the device, among them:
A. The vibrational unit, FIG. 3 (10).
B. An LED light display, FIG. 3 (2).
C. A sound source, FIG. 3 (12).
D. An electrophysiological stimulation unit, FIG. 3 (13).
E. A rhythm light display, FIG. 3 (4).
F. A timer.

The microprocessor may be programmed to:
A. Emit a set rhythm, with a choice of frequency from 8 to ½ cycles per second.
B. Emit any one of several rhythms in progression.
C. Sustain a rhythm for a time period ranging from less than a minute to more than an hour through the use of a timer.
D. Activate the rhythm at various times during the night.
E. Activate the vibrational unit only, so that the entire pad vibrates in synchrony with the chosen rhythm.
F. Activate the light source only, to pulse with the chosen rhythm.
G. Choose the intensity and the color of the light source.
H. Activate the sound function only, in synchrony with the rhythm, and regulate its volume.
I. Choose the type of sound emitted: tone, nature sounds, music, electronically synthesized sounds.
J. Activate the electrophysiological unit only, to emit bursts of microcurrent pulses to the pad in synchrony with the chosen rhythm, and to determine its output power. The microcurrents may be subliminal, below the level of awareness of the user, or may be para-subliminal. The duration of the bursts may be constant or variable and may be selected according to the user's preference or for therapeutic reasons. Current, measured in milliamperes (mA), may range from 1 to 100 mA. Electrical pulse width may range from 5 to 500 microseconds (μs). The pulse frequency may range from 1 or 2 pulses per second (pps) to 250 pps.
K. Activate any combination of the above modalities, including all of them, in unison.

Although an embodiment of the foregoing invention has been described in some detail by way of illustration and example for purposes of clarity of understanding, it will be obvious to those skilled in the art that certain changes and modifications may be practiced without departing from the spirit and scope thereof as described in the specification and as defined in the appended claims.

What is claimed is:

1. An apparatus for generating sleep-inducing stimuli, comprising in combination:
a programmable controller operable for generating a sleep-inducing rhythm; and
a transducer unit containing at least one transducer connected to said controller for receiving said sleep-inducing rhythm and generating and applying said sleep-inducing stimuli to a user in accordance with said rhythm.

2. The apparatus of claim 1, wherein said transducer unit is generally flat so as to be usable under the body of a sleeping user.

3. The apparatus of claim 1, wherein said transducer comprises one or more of: a vibrator, a localized light display, a multicolored light display, a spatially distributed light display, a sound generator, and an electrical current generator; each of which being disposed for transmitting said sleep-inducing rhythm to said user.

4. The apparatus of claim 3, wherein said electrical current generator in said transducer unit extends to a surface thereof for applying an electrical current to the body of said user in accordance with said rhythm.

5. The apparatus of claim 4, wherein said transducer unit has a conductive carbon silicone surface for contacting said user.

6. The apparatus of claim 4, wherein said current is approximately 1 to 100 ma.

7. The apparatus of claim 4, wherein said current is generated in the form of pulse bursts.

8. The apparatus of claim 7, wherein said pulses have a width of approximately 5 to 500 μs.

9. The apparatus of claim 7, wherein said pulses have a frequency of approximately 1 to 250 pps.

10. The apparatus of claim 3, wherein said multicolored display comprises a plurality of LED's.

11. The apparatus of claim 3, wherein said distributed light display comprises a plurality of LED's distributed on said transducer unit.

12. The apparatus of claim 1, wherein said controller is programmable for generating a constant rhythm.

13. The apparatus of claim 12, wherein said rhythm has a frequency of approximately 8 to 0.5 cps.

14. The apparatus of claim 1, wherein said controller is programmable for generating a varying rhythm.

15. The apparatus of claim 14, wherein said varying rhythm comprises at least two of: an alpha rhythm, a theta rhythm, and a delta rhythm, generated in that order.

16. The apparatus of claim 1, wherein said controller is programmable for generating said rhythm at one or a plurality of predetermined times.

17. A method of inducing sleep in a user, comprising the steps of:
juxtaposing a transducer unit with said user;
using a programmable controller to program a sleep-inducing rhythm;
supplying said sleep-inducing rhythm from said controller to a transducer in said transducer unit and thereby generating and applying sleep-inducing stimuli to said user.

18. The method of claim 17, wherein said transducer unit is placed under the body of the user.

19. The method of claim 17, wherein said stimuli comprise one or more of: vibrations, localized light, multicolored light, spatially distributed light, sound, and electrical current.

20. The method of claim 19, wherein said electrical current is approximately 1 to 100 ma.

21. The method of claim 19, comprising the step of generating said current in the form of pulse bursts.

22. The method of claim 21, wherein said pulses have a width of approximately 5 to 500 μs.

23. The apparatus of claim 21, wherein said pulses have a frequency of approximately 1 to 250 pps.

24. The method of claim 17, comprising the step of generating a constant rhythm.

25. The method of claim 24, wherein said rhythm has a frequency of approximately 12 to 0.5 cps.

26. The method of claim 17, comprising the step of generating a varying rhythm.

27. The method of claim 26, further comprising the step of generating at least two of: an alpha rhythm, a theta rhythm, and a delta rhythm, generated in that order.

28. The method of claim 17, comprising the step of generating said rhythm at one or a plurality of predetermined times.
